(19) United States
(12) Patent Application Publication
(10) Pub. No.: US 2023/0284955 A1
(43) Pub. Date: Sep. 14, 2023

(54) FLAT-BED KNIT-BASED ELECTRODE (HRV CHEST STRAP)

(71) Applicant: InDHouse Limited, Hong Kong (HK)

(72) Inventors: Tiffany Keesha Agathina WILLIAMS, Hong Kong (HK); Jackson Hoi Fung CHOW, Hong Kong (HK); Wen Ke XI, Hong Kong (HK)

(21) Appl. No.: 18/121,555

(22) Filed: Mar. 14, 2023

Related U.S. Application Data
(60) Provisional application No. 63/319,438, filed on Mar. 14, 2022.

(51) Int. Cl. A61B 5/282; A61B 5/00

(52) U.S. Cl. CPC A61B 5/282; A61B 5/6804

(57) ABSTRACT

Disclosed herein is a flat-bed knit-based electrode structure that has non-electrically conductive regions and electrically conductive regions. The non-electrically conductive regions are formed from a knitted textile including non-conductive yarns, and the electrically conductive regions are formed from a knitted textile having electrically conductive yarn. The electrically conductive regions are knitted using a conductive hybrid yarn containing a non-conductive multifilament with polymer and coated with carbon. The electrically conductive regions can transmit electrical data or power signals along the knitted textile via the conductive yarn. A connector links the conductive region to a wireless device that can output heart rate data of the user.

### CROSS-REFERENCE TO RELATED APPLICATIONS

This application claims the benefit of filing of U.S. Provisional App. No. 63/319,438 filed on Mar. 14, 2022, and which is incorporated by reference herein.

### FIELD OF THE INVENTION

The invention is generally for non-invasive measurement of heart rate information. More particularly, the present disclosure relates to heart rate monitors used in connection with exercise, sports and medical monitoring.

### BACKGROUND OF THE INVENTION

Prior art heart rate chest straps often use rigid electrode belts that can be difficult to fit around the chest comfortably. Moreover, long-term and continuous use of the electrode belt of prior art heart rate chest straps may cause chafing to the skin while training. What is needed is a wearable article or device that can integrate an electrode structure within the wearable article to permit heart rate monitoring without restricting the movement of or chafing the skin of a wearer.

### SUMMARY OF THE INVENTION

The present disclosure relates to an integrated flat-bed knit-based electrode structure and a heart rate measuring arrangement for measuring an electrocardiographic (ECG) signal detectable on or across the skin of a person's chest, arm or wrist. The flat-bed knit-based electrode structure comprises a band-like component that is fitted against or snugly conforms to the skin of the person's chest, arm or wrist and that is made of a soft, flat-bed knitted material with stretch recovery. The flat-bed knit-based electrode structure can further be configured with stitches including a texture, float, tuck, plaited, intarsia, or jacquard knit construction.

The flat-bed knit-based electrode structure may include electrically conductive fibers that are surrounded by an area of non-conductive textile fibers that can be defined with a wearable article, such as a chest strap, arm band, or wrist band. The wearable article can be configured to receive or transmit electrical signals to or from the wearer and also to or from a wireless electrical device. The heart rate is measured on a person's skin on the basis of an electrocardiographic (ECG) signal generated by a heartbeat.

The objective is to provide an electrically conductive region, which can function as a flat-bed knitted flexible electrode that can be integrated with a belt, band or other wearable articles of a conventional non-knitted or non-flexible construction. By placing a conductive area of the flat-bed knitted flexible electrode in close contact with the skin of the wearer, the electrically conductive region is able to detect electrical signals generated within the body of the wearer. Alternatively, such an electrode provides a point of contact on the skin to transmit to the wearer or wearer's skin an electrical signal generated externally to the wearer.

In some aspects, the present disclosure may provide a flat-bed knit-based electrode structure or “flexible electrode” system that can be incorporated into a wearable article, such as a chest strap, arm band, wrist band. The flat-bed knit-based electrode structure may provide an electrically conductive pathway or yarn for connection to a device for transmitting or receiving electrical signals to or from the body of the wearer. The flat-bed knit-based electrode structure may include non-electrically conductive areas having non-conductive yarns such as polyester or nylon and electrically conductive areas having electrically conductive yarns.

The flat-bed knit-based electrode structure may include two separate material portions that include electrically conductive regions. The electrically conductive regions can include a fabric having a textured, float, plaited, intarsia, or jacquard construction.

The flat-bed knit-based electrodes may be used in connection with or defined within a woven heart rate chest strap that can be connected to a measuring device. The measuring device can, for example, be used to monitor electrical signals of a wearer through the textile incorporating electrodes. For instance, the flat-bed knit-based electrodes can be used to receive monitoring of a wearer's ECG signal, which can be used to calculate, e.g., a wearer's heart rate or calories burned. Further illustrative embodiments show the knit-based electrodes may be incorporated into a chest strap to monitor the heart rate variability of the wearer during or after training. In the heart rate strap, the flat-bed knit-based electrodes may be incorporated into one band to be placed at the upper torso region or alternatively into a band for the wrist or upper arm region where a pulse can be detected. The wrist or arm band may include non-conductive areas and conductive areas, with portions of the skin contacting surface and the outer surface having electrically conductive yarns therein. Greater comfort is provided where the flat-bed knit-based electrodes are formed.

In other respects the invention relates to a flat-bed knit-based electrode structure for measuring an ECG signal on the skin of a person's chest, wrist or arm, wherein the electrode structure includes a band-like component having an inner flat-bed knitted conductive surface to be placed against the skin of the person's chest and an outer surface having electrically conductive fibers, the band-like component having a first electrically conductive region and a second electrically conductive region, wherein the electrode structure is arranged to measure a potential difference between the first and the second electrically conductive regions caused by the ECG signal.

The band-like component of the electrode structure may be a continuous band made of a flat-bed knitted textile that is a flexible, soft, and air permeable material that snugly conforms to the skin. The first electrically conductive region and the second electrically conductive region of the electrode structure may be electrically insulated from one another. The first electrically conductive region and the second electrically conductive region of the electrode structure may each form a conductive electrode. Each of the first electrically conductive region and the second electrically conductive region have a width that is less than a width of the band-like component. The electrode structure may comprise a monitoring unit in communication with the first electrically conductive region and the second electrically conductive region, wherein the monitoring unit receives ECG data from the first electrically conductive region and the second electrically conductive region and outputs heart rate information derived from the ECG data. When the monitoring unit is in connection with the first and second electrically conductive regions, the electrode structure may include one or more snaps for attaching the monitoring unit to the first and second electrically conductive regions of the electrode structure. The one or more snaps may be configured to attach the first and second electrically conductive regions to a surface of the monitoring unit casing which is against the person's skin and may form an electric coupling between the first and second electrically conductive regions and the monitoring unit. The electrode structure may be integrated into or defined within a wearable article, such as a chest strap, arm band, or wrist band.

### BRIEF DESCRIPTIONS OF THE DRAWINGS

FIG. 1 depicts a front view of a flat-bed knit-based electrode structure, according to an embodiment of the disclosure.

FIG. 1A depicts a front view, universal programming image of the non-electrically conductive permeable area of the flat-bed knit-based electrode of FIG. 1.

FIG. 11 depicts a front view, universal programming image of the electrically conductive area of the flat-bed knit-based electrode of FIG. 1.

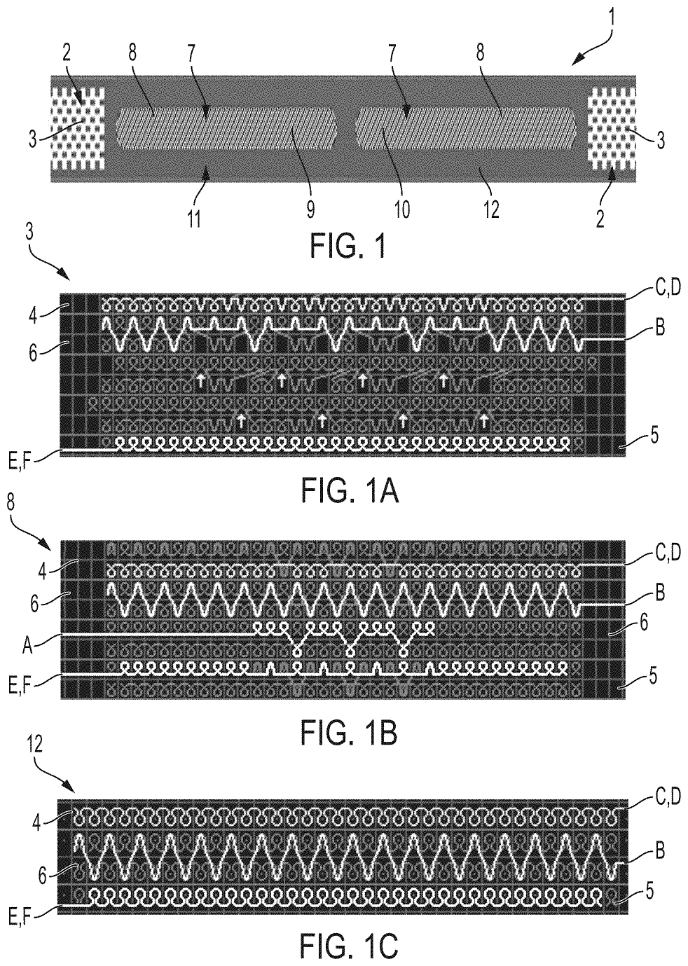

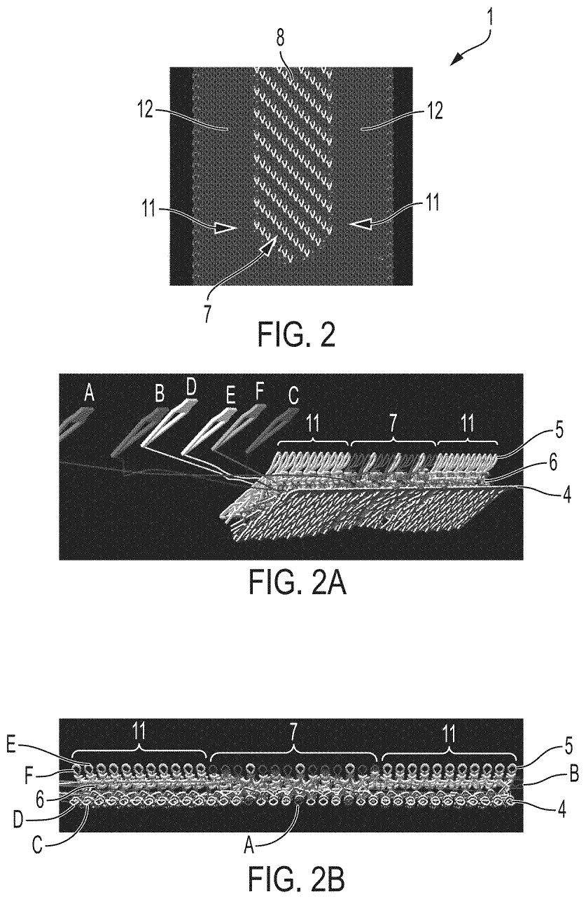

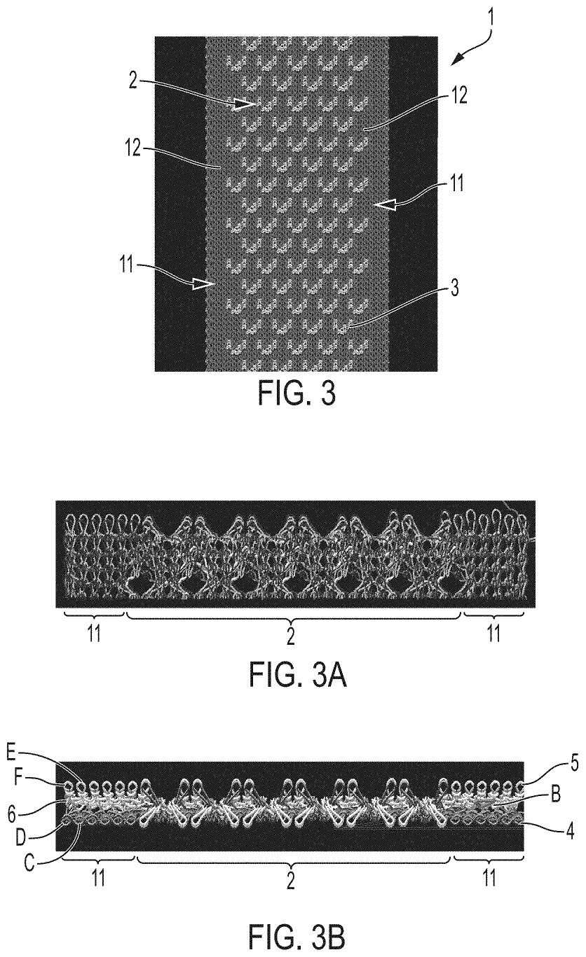

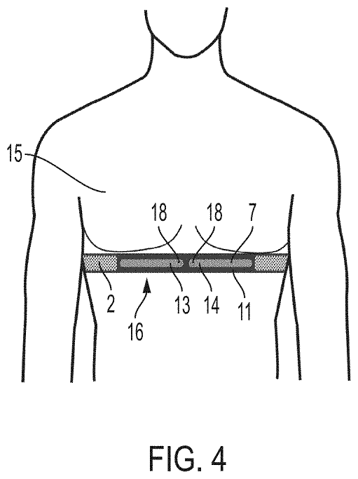

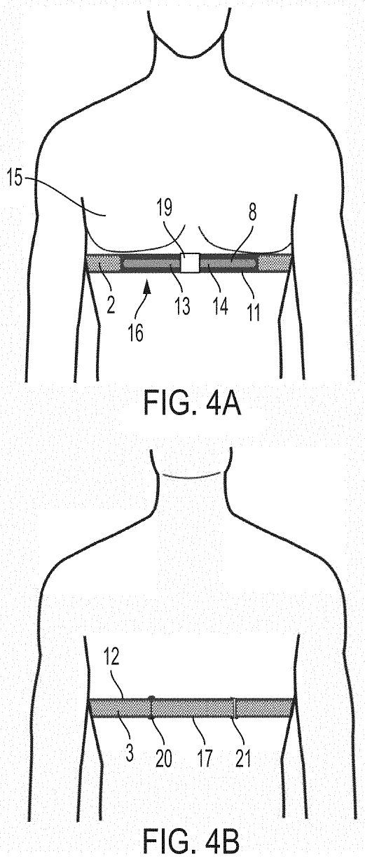

FIG. 1C depicts a front view, universal programming image of the non-electrically conductive area of the flat-bed knit-based electrode of FIG. 1.

FIG. 2 depicts an enlarged front view of a flat-bed knit-based electrode structure, according to an embodiment of the disclosure.

FIG. 2A depicts a perspective view, universal programming image of the flat-bed knit-based electrode of FIG. 2.

FIG. 2B depicts a cross-sectional view, universal programming image of the flat-bed knit-based electrode of FIG. 2.

FIG. 3 depicts another enlarged front view of a flat-bed knit-based electrode structure, according to an embodiment of the disclosure.

FIG. 3A depicts a front view, universal programming image of the wearable article of FIG. 3.

FIG. 3B depicts a stitch simulation universal programming image of the wearable article of FIG. 3.

FIG. 4 depicts a front view of an upper body wearable article incorporating a flat-bed knit-based electrode structure, according to an embodiment of the present disclosure.

FIG. 4A depicts another front view of the upper body wearable article of FIG. 4. In FIG. 4A, the upper body wearable article includes a monitoring unit.

FIG. 4B depicts a rear view of the upper body wearable article of FIG. 4A.

### DETAILED DESCRIPTION OF THE INVENTION

The present disclosure provides a flat-bed knit-based electrode structure 1 capable of being fully integrated into or defined within a wearable article that can be tailored to allow contact between the textile electrode and the body of the wearer. The flat-bed knit-based electrode structure 1 is used for the communication of information based on electrical signals provided by the electrode to the wearer of an article integrated with the textile electrode. As an example, the flat-bed knit-based electrode structure 1 may be adapted for the ECG monitoring of the wearer.

The flat-bed knit-based electrode structure 1 disclosed herein is also capable of transmitting or receiving electrical signals via contact with the body of the wearer without relying on bulky or delicate connection wires. In some embodiments the flat-bed knit-based electrode structure 1 may be communicated to a computing device using wireless technology (e.g., WiFi, BLUETOOTH®, etc.). The flat-bed knit-based electrode structure 1 may also be stretchable in the electrically conductive area due to the presence of elastic materials that are knitted with electrically conductive yarns or filaments. In this regard a flexible electrode structure may be more comfortable for the wearer to use.

Embodiments falling within the scope of the present invention are further described with references to the figures disclosed herein.

In one embodiment, the flat-bed knit-based electrode structure 1 is provided with a non-electrically conductive, permeable area 2 comprising a first fabric 3. The first fabric 3 is permeable for airflow. The first fabric 3 includes yarns B-F, as represented in FIG. 1A. In the non-electrically conductive, permeable area 2, the first fabric 3 is formed using a double bed knitting technique with transfer of yarn to the neighbouring needles for two courses, creating a hole on the front and back of the fabric. The first fabric is prepared using a knitting machine having a front needle bed 4 and a back needle bed 5. The knitting machine shifts the first fabric 3 from the front needle bed 4, including yarns C and D, to the back needle bed 5, including yarns E and F. In the same course there is a central area 6 including a knitting tuck stitch in yarn B, using a 1×1 needle (1×1 is every other needle). In other words, Yarn B is incorporated using a knitting tuck stitch every other needle in Lycra® Spandex for every knitting course. This technique increases the stretch recovery of the fabric. For the plaited area, the front needle bed 4 has yarns C and D in the same feeder and the back needle bed 5 has yarns E and F in the same feeder. Yarn C is front facing and yarn D faces into the central area 6 of the knit. Yarn E is front facing and yarn F faces into the central area 6 of the knit.

The front needle bed 4, back needle bed 5, and central area 6 are knitted together simultaneously in each knitting course. In the first fabric 3, the electrically conductive yarn A is not present.

The flat-bed knit-based electrode structure 1 also includes an electrically conductive area 7 comprising a second fabric 8 formed from electrically conductive yarn A and non-electrically conductive yarns B-F, which are electrically isolated from the first fabric 3, as represented in FIG. 1B. Yarn A may be a conductive yarn. For example, Yarn A may be a conductive hybrid yarn having a non-conductive multifilament with polymer and coated with carbon. The electrically conductive area 7 may use an intarsia and/or jacquard knit construction. The electrically conductive area 7 may be divided into two or more electrode regions formed of the second fabric 8. For example, the electrically conductive area 6 may include a first electrically conductive region 9 (“Electrode Block 1”) and a second electrically conductive region 10 (“Electrode Block 2”). The first and second electrically conductive regions 9,10 may be completely separate regions that do not connect with each other or overlap. The first electrically conductive region and the second electrically conductive region may each form a conductive electrode, as discussed below.

In some embodiments, the flat-bed knit-based electrode structure may include a non-electrically conductive area 11 comprising a third fabric 12 that is isolated on the borders of the non-conductive, permeable area 2 and in-between the first and second electrically conductive regions 9, 10. The third fabric 12 includes yarns B-F, as represented in FIG. 1C. The third fabric 12 is formed using a double bed knitting technique compromised of plaiting with transfer knitting shifting from front needle bed 4, including yarns C and D, to the back needle bed 5, including yarns E and F. In the same course there is central area 6 including a knitting tuck stitch in yarn B, 1×1 needle (1×1 is every other needle). Yam B is incorporated using a knitting tuck stitch every other needle in Lycra® Spandex for every knitting course, adding to the stretch recovery. For the plaited area, the front needle bed 4 has yarns C and D in the same feeder and the back needle bed 5 has yarns E & F in the same feeder. Yarn C is front facing and yarn D faces into the central area 6 of the knit. Yarn E is front facing and yarn F faces into the central area 6 of the knit. The front needle bed 4, back needle bed 5, and central area 6 are woven together simultaneously. In the third fabric 12, the portion of electrically conductive yarn A is not present.

FIG. 2 shows a section of the flat-bed knit-based electrode structure 1 including an electrically conductive area 7 and non-electrically conductive area 11. FIGS. 2A and 2B show a side view and a cross-sectional view, respectively, of the section of the flat-bed knit-based electrode structure 1 of FIG. 2 including the second fabric 8 and third fabric 12. FIGS. 2A and 2B are universal programming images showing the yarn location set up, central area 6, and front needle bed 4. Also visible in FIGS. 2A and 2B are various stitch combinations of plaiting, tuck stitch, intarsia, and jacquard.

Similarly, FIG. 3 shows a section of the flat-bed knit-based electrode structure 1 including a non-electrically conductive, permeable area 2 and a non-electrically conductive area 11. The section of the flat-bed knit-based electrode structure 1 of FIG. 3 is permeable to permit airflow and formed using a double bed knitting technique. This section of the flat-bed knit-based electrode structure 1 is also non-electrically conductive. FIGS. 3A and 3B show a front view and a cross-sectional view, respectively, of the section of the flat-bed knit-based electrode structure 1 of FIG. 3 including the first fabric 3 and the third fabric 12. FIGS. 3A and 3B are universal programming images showing the yarn location set up, central area 6, and front needle bed 4. Also visible in FIGS. 3A and 3B are various stitch combinations of plaiting, tuck stitch, and permeable Pointelle area.

FIG. 4 shows a heart rate measuring arrangement of the flat-bed knit-based electrode structure 1 placed on a wearer's chest 15 according to an embodiment of the invention. The electrode structure 1 is in the form of a band-like component 16 that is a continuous band having an inner surface against the skin of the wearer's chest 15 and an outer surface 17 which is opposite thereto. The width of each of the first and second electronically conductive regions 9, 10 may be less than the width of the band-like component 16. ECG signals are identified by means of the flat-bed knit-based electrode structure 1 placed on the chest 15 in order to determine or measure the heart rate. In particular, the heart rate is measured by means of first and second electrodes 13, 14 formed by the first and second electrically conductive regions 9, 10 in the electrode structure 1. The first electrode 13 and the second electrode 14 of the electrode structure 1 are electrically separated from one another by a section of third fabric 12 disposed between the electrodes, which enables the measurement of the potential difference between the electrodes 13, 14 based on the heart beats. A measurable potential difference is therefore produced between the first electrode 13 and the second electrode 14, which may be, for example, an ECG signal that is measured with the electrode structure 1.

Referring next to the snap 18, one or more flat-bed knit-based electrodes 13,14 may be formed in the band-like component 16 and have one or more electrically conductive snaps 18 for electrically coupling the first and second electrodes 13, 14 to an electrical device such as a monitoring unit 19. The monitoring unit 19 is a computer processing unit (CPU) programmed to receive the electrical signal information from the first and second electrically conductive regions 9, 10 and determine recognizable information, such as the wearer's heart rate or calories burned from exercise or other activity. The monitoring unit 19 may be in communication with a device such as a remote computer, a monitor, a smartphone, etc. for providing the recognizable information to the wearer or to another person (e.g., a doctor or trainer).

The electrically conductive snaps 18 are attached to the first and second electrodes 13, 14. The electrically conductive snaps 18, may be made of any electrically conductive material, such as a metallic conductor. In some embodiments, the snaps 18 may connect to a surface of the monitoring unit 19 that is configured to contact a wearer's skin. In other embodiments, the snaps 18 may connect to leads of a monitoring unit 19. When snaps 18 electrically couple the first and second electrodes 13, 14 to a device such as a monitoring unit 19 as shown in FIG. 4A, physical characteristics within the body, such as heart rate or ECG, may be transmitted to the device. FIG. 4B is the back view of the band-like component 16 which includes the first fabric 3 and the third fabric 12, in addition to metal or plastic accessories to complete the strap detail, such as a ring slider 20 and G hook 21.

Even though the invention is described above with reference to the examples of the attached drawings, it is apparent that the invention is not restricted thereto but it can be modified via stitch and yarn in a variety of ways within the scope of the inventive idea disclosed in the accompanying claims.

1. A flat-bed knit-based electrode structure for measuring an ECG signal on the skin of a person's chest, wrist or arm, wherein the electrode structure comprises:
a band-like component having an inner flat-bed knitted conductive surface to be placed against the skin of the person's chest and an outer surface having electrically conductive fibers, the band-like component having
a first electrically conductive region and
a second electrically conductive region,
wherein the electrode structure is arranged to measure a potential difference between the first and the second electrically conductive regions caused by the ECG signal.

2. The electrode structure of claim 1, wherein the band-like component of the electrode structure is a continuous band made of a flat-bed knitted textile that is a flexible, soft and air permeable material that fits the skin closely.

3. The electrode structure of claim 1, wherein the first electrically conductive region and the second electrically conductive region of the electrode structure are electrically insulated from one another.

4. The electrode structure of claim 1, wherein the first electrically conductive region and the second electrically conductive region of the electrode structure each form a conductive electrode.

5. The electrode structure of claim 1, wherein each of the first electrically conductive region and the second electrically conductive region have a width that is less than a width of the band-like component.

6. The electrode structure of claim 1, wherein the electrode structure comprises a monitoring unit in communication with the first electrically conductive region and the second electrically conductive region, wherein the monitoring unit receives ECG data from the first electrically conductive region and the second electrically conductive region and outputs heart rate information derived from the ECG data.

7. The electrode structure as claimed in claim 6, wherein the monitoring unit is in connection with the first and second electrically conductive regions, wherein the electrode structure comprises one or more snaps for attaching the monitoring unit to the first and second electrically conductive regions of the electrode structure.

8. The electrode structure as claimed in claim 7, wherein the one or more snaps are configured to attach the first and second electrically conductive regions to a surface of the monitoring unit casing which faces the person's skin.

9. The electrode structure as claimed in claim 7, wherein the one or more snaps form an electric coupling between the first and second electrically conductive regions and the monitoring unit.

10. The electrode as claimed in claim 1, wherein the electrode structure is integrated into a wearable article, such as a chest strap, arm band or wrist band.
